(19) United States
(12) Patent Application Publication
(10) Pub. No.: US 2025/0090022 A1
(43) Pub. Date: Mar. 20, 2025

(54) OPHTHALMIC EXAMINATION SYSTEM, AND PHYSIOLOGICAL INFORMATION CAPTURE DEVICE AND OPHTHALMIC EXAMINATION METHOD THEREOF

(71) Applicant: PolyVisions Technology Co., Ltd., NEW TAIPEI CITY (TW)

(72) Inventors: Chih-Ju Lin, NEW TAIPEI CITY (TW); Shih-Chieh Yen, NEW TAIPEI CITY (TW); Chen-Fu Huang, NEW TAIPEI CITY (TW)

(73) Assignee: PolyVisions Technology Co., Ltd., NEW TAIPEI CITY (TW)

(21) Appl. No.: 18/636,701

(22) Filed: Apr. 16, 2024

(30) Foreign Application Priority Data
Sep. 14, 2023 (CN) 202311186871.2

(51) Int. Cl. A61B 3/14; A61B 3/103; A61B 3/12

(52) U.S. Cl. CPC A61B 3/145; A61B 3/103; A61B 3/12

(57) ABSTRACT

An eye detection physiological information capture device includes a first light plate assembly, and a second light plate assembly arranged in a housing. The first light plate assembly includes a first light source module, and a signal detection module. The signal detection module detects the reflection of light emitted from the first light source module towards an eye. The second light plate assembly includes a second light source module, a lens assembly, a first camera module, and a second camera module. The lens assembly is arranged to enable light rays from the second light source module to respectively irradiate the eye. The first camera module can capture a three-dimensional image of the eye and the second camera module can capture a fundus image of the eye by detecting the reflection of light emitted from the second light source module towards the eye.

### CROSS-REFERENCE TO RELATED APPLICATION

This non-provisional application claims priority under 35 U.S.C. § 119(a) to Patent Application No. 202311186871.2 filed in China on Sep. 14, 2023, the entire contents of which are hereby incorporated by reference.

### BACKGROUND

### Technical Field

The present invention relates to a detection system for an eye, and in particular, to a capture device for capturing physiological information and a detection method thereof.

### Related Art

In recent years, it is common to detect physiological conditions of human bodies by using non-invasive instruments. The non-invasive instruments generally have the advantage of executing tasks more quickly, more conveniently, and safer compared to a conventional invasive detection method, which reduces the time waiting and complex assays, and reduces physical stimulation and infection risks because of instruments entering patients' bodies. Although the non-invasive physiological detection instruments provide less accurate results than the conventional biochemical detection manner, the non-invasive physiological detection instruments can provide specific reference values and provide specific assistance for early physiological monitoring.

Most measurements of non-invasive instruments are adopted optical technologies. Optical technologies can be applied to many aspects regarding physiological monitoring and medical tests, including brain image and the monitors of blood pressure, heart rate, breath, and blood oxygen, and the like. In addition, optical measurement is suitable for patients of different ages, postures, and health conditions. The applied optical methods will be depended on the types of detecting different physiology and materials of body, such as the photon absorption, scattering, and image reflection. Optical technologies will be widely used to develop the hospital and home using medical devices.

For example, most of the common smart watches on the market are equipped with light sources with specific wavelengths to detect the physiological conditions such as blood oxygen and heart rate. Users only need to wear a smart watch on wrists, to monitor pulse oxygen saturation (SpO2) via an optical technology, which can detect the blood oxygen saturation based on the absorptions of infrared and visible light.

### SUMMARY

In view of the above, the present invention provides a physiological information capture device for an eye, an ophthalmic examination system, and an ophthalmic examination method, to detect the different ophthalmic and physiological information through light sources with multi bands and capturing modules.

In an embodiment, the physiological information capture device for an eye includes a housing, a first light plate assembly, and a second light plate assembly. The housing includes a capturing side and a mounting side. The capturing side and the mounting side are opposite to the other. The first light plate assembly is arranged in the housing and located close to the capturing side. The first light plate assembly includes a first circuit board, a first light source module, and a signal detection module. The first circuit board is provided with a first hole. The first light source module is located on the first circuit board and surrounds the first hole. The first light source module includes a plurality of first light-emitting elements. The signal detection module is located on the first circuit board. The signal detection module is provided with a plurality of signal detectors corresponding to the plurality of first light-emitting elements. The second light plate assembly is arranged in the housing and located close to the mounting side. The second light plate assembly includes a second circuit board, a second light source module, a lens assembly, first camera modules, and a second camera module. The second circuit board is provided with a second hole. The second hole is aligned with the first hole. A diameter of the first hole is greater than the second one. The second light source module is located on the second circuit board. The second light source module includes a group of inner light-emitting elements and at least one group of outer light-emitting elements. The inner light-emitting elements surround the second hole. The at least one group of outer light-emitting elements is concentrically arranged with respect to a center of the second hole and surrounds the inner light-emitting elements and the second hole. The lens assembly is arranged in the housing. A lens optical axis thereof is aligned with the center of the second hole. The lens assembly includes a first lens. The first lens covers the inner light-emitting elements and the second hole. The plurality of first camera modules are located on a periphery of the second hole. A camera optical axis (COA) of each of the first camera modules defines an included angle relative to a normal line of an upper surface of the second circuit board. A COA of the second camera module is aligned with the center of the second hole.

In an embodiment, the first light source module includes a plurality of first light source groups with different wavelengths. Each of the first light source groups includes a plurality of first light-emitting elements with the same wavelength.

In an embodiment, each of the first light-emitting elements generates a first light ray to irradiate a sclera of an eye. The plurality of signal detectors receive first reflected light of the first light ray reflected from the sclera of the eye.

In an embodiment, the inner light-emitting elements to generate a second light ray to irradiate an iris and a fundus of the eye. The pluralities of groups of outer light-emitting elements generate a third light ray to irradiate a pupil of the eye. The first camera module receives the first reflected light of the first light ray reflected from the sclera of the eye, a second reflected light of the second light ray reflected from the iris of the eye, and third reflected light of the third light ray reflected from the fundus of the eye.

In an embodiment, the second camera module receives the second reflected light of the second light ray reflected from the fundus/retina of the eye.

In an embodiment, wavelengths of the inner light-emitting elements are different from wavelengths of the at least one group of outer light-emitting elements.

The present invention further provides an ophthalmic examination system, which includes a physiological information capture device, a processor, a first display, and a second display. Each first light-emitting element of the physiological information capture device generates a first light ray to irradiate a sclera of an eye. Inner light-emitting elements generate a second light ray to irradiate an iris and a fundus of the eye. At least one group of outer light-emitting elements generates a third light ray to irradiate a pupil of the eye. A plurality of signal detectors receive first reflected light of the first light ray reflected from the sclera and convert the first reflected light into a physiological signal. First camera modules receive the first reflected light reflected from the sclera, second reflected light of the second light ray reflected from the iris, and third reflected light of the third light ray reflected from the fundus of the eye, and respectively convert the first reflected light, the second reflected light, and the third reflected light into a three-dimensional eye surface signal, an iris signal, and a dioptric signal. A second camera module receives the second reflected light of the second light ray reflected from the fundus and convert the second reflected light into a fundus signal. The processor is coupled to the physiological information capture device, and the processor: process the physiological signal and convert the physiological signal into a physiological value, process the three-dimensional eye surface signal and convert the three-dimensional eye surface signal into a three-dimensional eye surface image, process the dioptric signal and convert the dioptric signal into a diopter value, process the fundus signal and convert the fundus signal into a fundus image, and receive the iris signal. The first display is coupled to the processor and displays a group composed of the physiological value, the two-dimensional eye surface image, the diopter value, the fundus image, and a combination thereof. The second display is coupled to the processor and displays the three-dimensional eye surface image.

The present invention further provides an ophthalmic examination method, which includes the following steps: receiving a detection instruction; starting a first light source module or a second light source module based on the detection instruction, where the first light source module generates a first light ray, and the second light source module generates a second light ray and a third light ray; performing the following steps when the first light source module is started: starting a signal detection module or a first camera module based on the detection instruction; receiving first reflected light of the first light ray reflected from a sclera of an eye; converting the first reflected light into a physiological signal and converting the physiological signal into a physiological value when the signal detection module is started; and converting the first reflected light into a three-dimensional eye surface signal and converting the three-dimensional eye surface signal into a two-dimensional eye surface image and a three-dimensional eye surface image when the first camera module is started; displaying the physiological value or the two-dimensional eye surface image and the three-dimensional eye surface image; and performing the following steps when the second light source module is started: starting the first camera module or a second camera module based on the detection instruction; receiving third reflected light of the third light ray reflected from a fundus of the eye, converting the third reflected light into a dioptric signal, converting the dioptric signal into a diopter value, and displaying the diopter value, when the first camera module is started; and receiving second reflected light of the second light ray reflected from the fundus of the eye, converting the second reflected light into a fundus signal, converting the fundus signal into a fundus image, and displaying the fundus image, when the second camera module is started.

The present invention is described in detail below with reference to drawings and specific embodiments, which are not construed as a limitation on the present invention.

### BRIEF DESCRIPTION OF THE DRAWINGS

FIG. 1 is a three-dimensional schematic diagram of a physiological information capture device according to an embodiment.

FIG. 2 is a three-dimensional schematic diagram of a physiological information capture device according to another embodiment.

FIG. 3 is a schematic diagram of the embodiment of FIG. 1 when used on an eye.

FIG. 4 is a three-dimensional schematic diagram of a first light plate assembly and a second light plate assembly in the embodiment of FIG. 1.

FIG. 5A is a top view of a first light plate assembly according to an embodiment.

FIG. 5B is a top view of the first light plate assembly according to another embodiment.

FIG. 6A is a top view of an example of a second light source module in a second light plate assembly.

FIG. 6B is a top view of another example of the second light source module in the second light plate assembly.

FIG. 7 is a cross-sectional view of a position A-A marked in FIG. 4.

FIG. 8 is a top view of a second light plate assembly according to an embodiment.

FIG. 9 is a three-dimensional schematic diagram of a physiological information capture device according to still another embodiment, showing that the physiological information capture device includes a rotation module.

FIG. 10 is a three-dimensional schematic diagram of an example of the rotation module in FIG. 9.

FIG. 11 is a block diagram of an ophthalmic examination system according to an embodiment.

FIG. 12 is a flowchart of detecting a physiological value and an eye surface image of an ophthalmic examination method according to an embodiment.

FIG. 13 is a flowchart of detecting a diopter value and a fundus image in an ophthalmic examination method according to an embodiment.

FIG. 14 is a flowchart of performing an anomaly marking process in an ophthalmic examination method according to an embodiment.

FIG. 15 is a flowchart of iris recognition in an ophthalmic examination method according to an embodiment.

### DETAILED DESCRIPTION

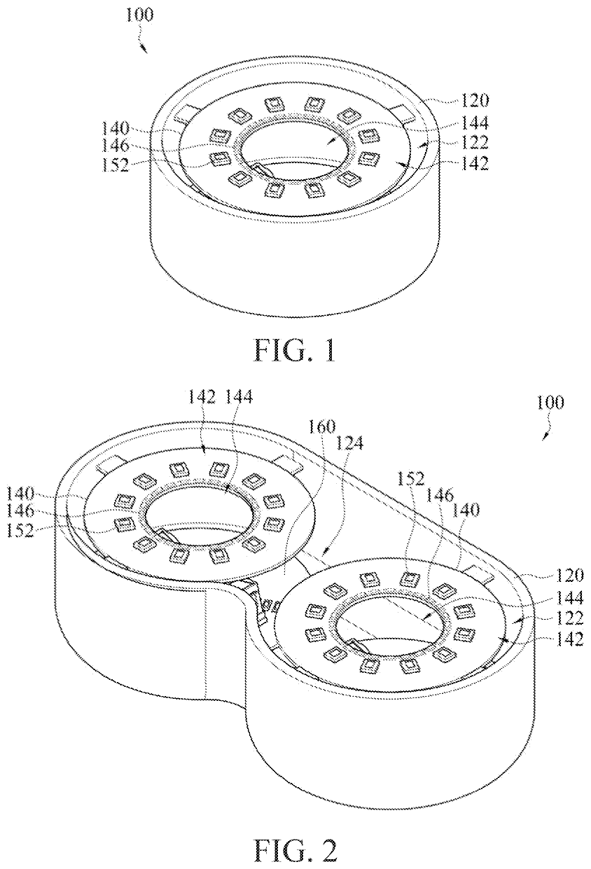

Referring to FIG. 1 and FIG. 2, FIG. 1 is a three-dimensional schematic diagram of a physiological information capture device according to an embodiment, and FIG. 2 is a three-dimensional schematic diagram of a physiological information capture device according to another embodiment. The physiological information capture device 100 includes a housing 120, a first light plate assembly 140, and a second light plate assembly 160. The physiological information capture device 100 may be designed for use on a single eye or both eyes. When the physiological information capture device is designed for use on a single eye, The housing 120 is in a shape of a cylinder. Two ends of the cylinder are opposite to each other. The two ends are respectively a capturing side 122 and a mounting side 124 of the housing 120. When a user uses the physiological information capture device 100, the capturing side 122 is a side closer to eyes of the user.

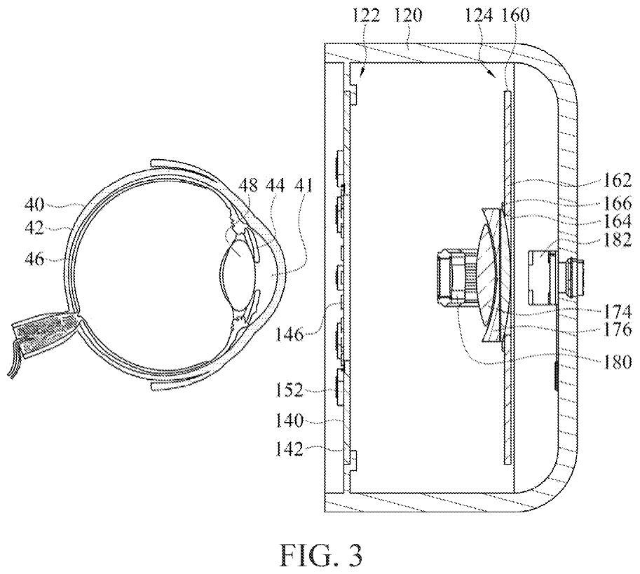

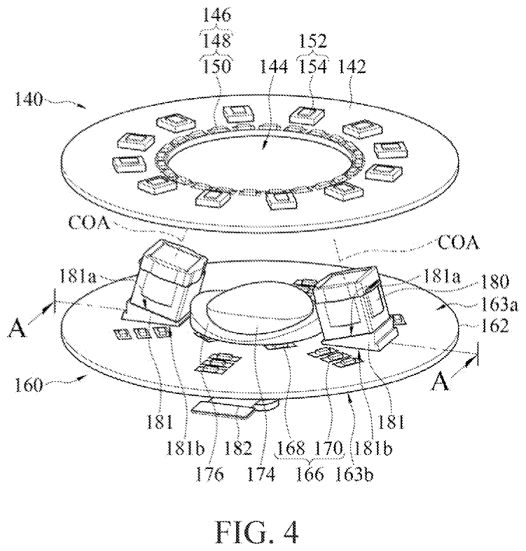

Referring to FIG. 3 and FIG. 4, FIG. 3 is a schematic diagram of the embodiment of FIG. 1 when used on an eye, and FIG. 4 is a three-dimensional schematic diagram of a first light plate assembly and a second light plate assembly in the embodiment of FIG. 1. A first light plate assembly 140 of the physiological information capture device 100 is arranged in the housing 120 and located close to the capturing side 122. A second light plate assembly 160 is also arranged in the housing 120 but is located close to the mounting side 124. When the physiological information capture device 100 is designed for use on both eyes, the housing 120 is a combination of two parallel cylinders. The two cylinders are connected by a connecting structure that spans a nose. The two cylindrical shapes each have the capturing side 122 and the mounting side 124. The first light plate assembly 140 and the second light plate assembly 160 are respectively located close to the capturing side 122 and close to the mounting side 124. When a user uses the physiological information capture device 100, a distance between an eye surface and the first light plate assembly 140 may be less than one centimeter.

The first light plate assembly 140 includes a first circuit board 142, a first light source module 146, and a signal detection module 152. The first circuit board 142 is provided with a first hole 144. A center of the first hole 144 corresponds to a center of the first circuit board 142. The first light source module 146 is located on the first circuit board 142 and surrounds the first hole 144. The first light source module 146 includes a plurality of first light-emitting elements 150.

The signal detection module 152 is located on the first circuit board 142. The signal detection module 152 is provided with a plurality of signal detectors 154 corresponding to the plurality of first light-emitting elements 150. The expression “corresponding to” means that spectral ranges that can be measured by the signal detectors 154 cover wavelengths of the first light-emitting elements 150.

The second light plate assembly 160 includes a second circuit board 162, a second light source module 166, a lens assembly 174, a plurality of first camera modules 180, and a second camera module 182. The second circuit board 162 is provided with a second hole 164. The second hole 164 is aligned with the first hole 144. A diameter of the first hole 144 is greater than a diameter of the second hole 164.

The second light source module 166 is located on the second circuit board 162. The second light source module 166 includes a group of inner light-emitting elements 168 and at least one group of outer light-emitting elements 170. A “group” may include a plurality of inner light-emitting elements 168 or a plurality of outer light-emitting elements 170. For example, four inner light-emitting elements 168 may form a group of inner light-emitting elements 168, and six outer light-emitting elements 170 may form a group of outer light-emitting elements 170. The group of inner light-emitting elements 168 surround the second hole 164. The at least one group of outer light-emitting elements 170 are concentrically arranged with respect to the second hole 164 and surround the group of inner light-emitting elements 168 and the second hole 164.

The lens assembly 174 is arranged in the housing 120, and the lens assembly 174 has a lens optical axis aligned with a center of the second hole 164. The lens assembly 174 includes a first lens 176. The first lens 176 covers the inner light-emitting elements 168 and the second hole 164.

The plurality of first camera modules 180 are located on a periphery of the second hole 164. In some embodiments, two first camera module 180 are arranged. The two first camera modules 180 are respectively located on two sides of the second hole 164. The first camera modules 180 have respective camera optical axes (COAs). Each COA defines an included angle θ_{cam} with respect to a normal line Ln of an upper surface 163a of the second circuit board 162 (further refer to FIG. 7), which may be realized through a shape of a fixing post 181 on which the first camera module 180 is arranged. For example, the fixing post 181 is a shape of a triangular post (refer to FIG. 4). One side of the triangular post is used as an upper fitting surface 181a of the fixing post 181, and another side is used as a lower fitting surface 181b of the fixing post 181. An angle formed between the upper fitting surface 181a and the lower fitting surface 181b is the same as the included angle θ_{cam}. The upper fitting surface 181a is fixed to the first camera module 180, and the lower fitting surface 181b is attached to the upper surface 163a of the second circuit board 162. In this way, the COA defines the included angle θ_{cam} relative to the normal line Ln of the upper surface 163a. In some embodiments, the included angle θ_{cam} is 20 degrees. A COA of the second camera module 182 is aligned with the center of the second hole 164.

In some embodiments, the diameter of the first hole 144 is 30 millimeters. In this case, when the first light source module 146 surrounds the first hole 144, a diameter of a circle formed by the first light source module 146 is greater than a general diameter of a pupil 41 of an eye 40. However, in a case that the diameter of the second hole 164 is less than the diameter of the first hole 144, a light ray generated by the second light source module 166 can pass through the first hole 144 without being blocked by the first circuit board 142. In addition, a distance between the second circuit board 162 and the first circuit board 142 is 8 centimeters. In this case, when the included angle θ_{cam} defined by the COA of the first camera module 180 relative to the normal line Ln of the upper surface 163a of the second circuit board 162 is 20 degrees, an imaging light ray entering the first camera module 180 can also pass through the first hole 144 without being blocked by the first circuit board 142.

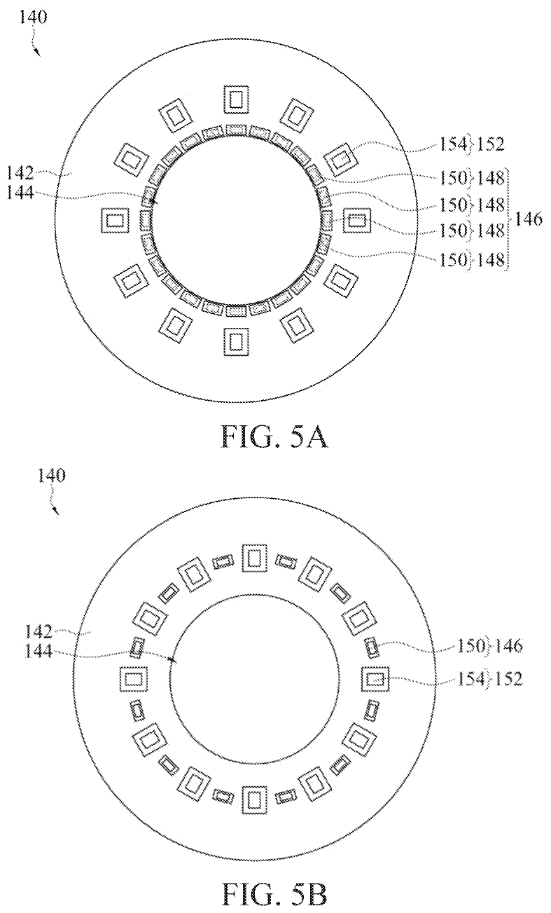

Referring to FIG. 5A, FIG. 5A is a top view of an example of a first light plate assembly. In some embodiments, the plurality of first light-emitting elements 150 included in the first light source module 146 generate first light rays. The first light rays with a same wavelength.

In some other embodiments, the first light source module 146 includes a plurality of first light source groups 148. Each of the first light source groups 148 includes a plurality of first light-emitting elements 150. The first light rays generated by the first light source groups 148 with different wavelengths, while the first light rays generated by the first light-emitting elements 150 within the first light source group 148 with the same wavelength.

The wavelengths of the first light rays may be between a wavelength of visible light and a wavelength of infrared light. For example, the first light source module 146 includes a plurality of first light source groups 148, and first light rays generated by the first light-emitting elements 150 in each of the first light source groups 148 with a same wavelength. In some embodiments, wavelengths of the first light rays generated by the plurality of first light source groups 148 may be light sources of 460 nanometers (visible light), 660 nanometers (visible light), 940 nanometers (infrared light), 1100 nanometers (infrared light), 1350 nanometers (infrared light), and the like. A first light-emitting element 150 of 460 nanometers, a first light-emitting element 150 of 660 nanometers, a first light-emitting element 150 of 940 nanometers, a first light-emitting element 150 of 1100 nanometers, and a first light-emitting element 150 of 1350 nanometers are cyclically arranged in sequence.

In still some other embodiments, the first light source module 146 includes a plurality of first light source groups 148. Each of the first light source groups 148 includes a plurality of first light-emitting elements 150 that may generate first light rays within different wavelengths. For example, a first light source group 148 includes five first light-emitting elements 150. The wavelengths of the first light rays generated by the five first light-emitting elements 150 are respectively 460 nanometers, 660 nanometers, 940 nanometers, 1100 nanometers, and 1350 nanometers. The first light source groups 148 are adjacent to each other and surround the first hole 144 to form the first light source module 146.

Different wavelengths of the first light rays may correspond to different physiological substances. For example, the first light ray of 460 nanometers corresponds to a measurement of bilirubin, the first light ray of 660 nanometers corresponds to a measurement of blood oxygen saturation, the first light ray of 940 nanometers corresponds to a measurement of total heme, the first light ray of 1100 nanometers correspond to a measurement of cholesterol, and the first light ray of 1350 nanometers corresponds to a measurement of glucose. These first light rays irradiate a sclera 42 of the eye 40. The signal detector 154 receives first reflected light of the first light rays reflected from the sclera 42. The signal detector 154 converts the received optical signals into electrical signals and transmits the signals to a processor 200 for calculation (which is detailed later), to obtain information about the foregoing physiological substances.

In this embodiment, corresponding to the wavelengths of the first light rays generated by the first light source group 148, detection ranges of the plurality of signal detectors 154 of the signal detection module 152 may be divided into two groups. The detection ranges of one group of signal detectors 154 cover the visible light, and the detection ranges of the other group of signal detectors cover the infrared light. The signal detectors 154 with different detection ranges are arranged alternately and surround a periphery of the first light source module 146.

Referring to FIG. 5B, FIG. 5B is a top view of another example of the first light plate assembly. In some other embodiments, the plurality of signal detectors 154 and the plurality of first light-emitting elements 150 for a circle, and the signal detectors 154 and the first light-emitting elements 150 are arranged alternately.

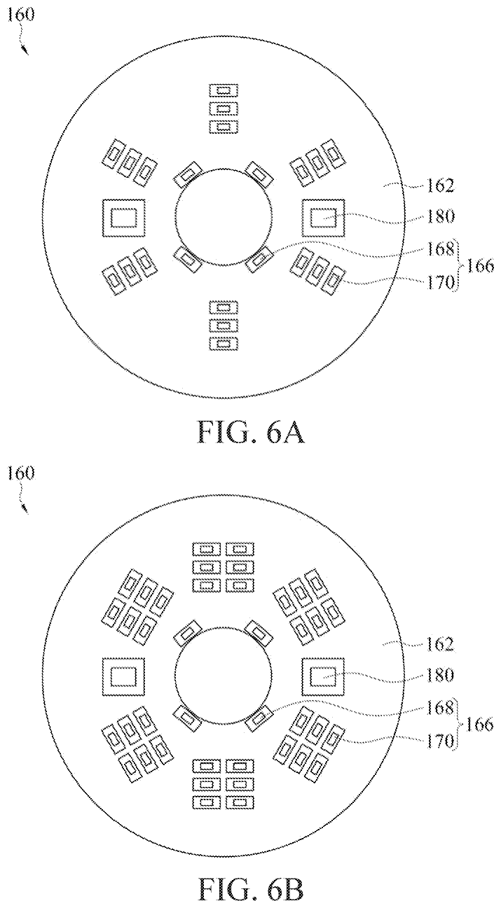

Referring to FIG. 6A, FIG. 6A is atop view of an example of a second light source module in a second light plate assembly. The at least one group of outer light-emitting elements 170 may include three groups of outer light-emitting elements. As described above, a “group” may include a plurality of inner light-emitting elements 168 or a plurality of outer light-emitting elements 170. A quantity of inner light-emitting elements 168 included in a group of inner light-emitting elements 168 may be the same or different from a quantity of outer light-emitting elements 170 included in a group of outer light-emitting elements 170, which may be adjusted as required.

The plurality of inner light-emitting elements 168 and the plurality of outer light-emitting elements 170 are respectively evenly distributed at all angles of the second hole 164. For example, a plurality of inner light-emitting elements 168 included in a group of inner light-emitting elements 168 are respectively distributed in four directions of the second hole 164, and a plurality of outer light-emitting elements 170 included in a group of outer light-emitting elements 170 are respectively distributed in six directions of the second hole 164. The four directions may be respectively located at 0 degrees, 90 degrees, 180 degrees, and 270 degrees of the second hole 164. The six directions may be respectively located at 0 degrees, 60 degrees, 120 degrees, 180 degrees, 240 degrees, and 300 degrees of the second hole 164.

For example, a group of inner light-emitting elements 168 is formed by arranging one inner light-emitting element 168 is arranged in each of four directions. A quantity of inner light-emitting elements 168 included in the group of inner light-emitting elements is four. For example, a group of outer light-emitting elements 170 is formed by arranging one outer light-emitting element 170 in each of six directions. A quantity of outer light-emitting elements 170 included in the group of outer light-emitting elements is six. When three groups of outer light-emitting elements 170 are concentrically arranged around the second hole 164, a total quantity of outer light-emitting elements 170 included in the three groups of outer light-emitting elements is 18 (as shown in FIG. 6A).

Referring to FIG. 6B, FIG. 6B is atop view of another example of the second light source module in the second light plate assembly. In some embodiments, if a group of outer light-emitting elements 170 is formed by arranging two outer light-emitting elements 170 (for example, arranged side by side) in each of six directions, a quantity of outer light-emitting elements 170 included in the group of outer light-emitting elements 170 is twelve. If three groups of outer light-emitting elements 170 are concentrically arranged around the second hole 164, a total quantity outer light-emitting elements 170 included in the three groups of outer light-emitting elements is 36.

In some embodiments, each of the inner light-emitting elements 168 generates a second light ray, and each of the outer light-emitting elements 170 generates a third light ray. A wavelength of the second light ray is different from that of the third light ray. The wavelengths of the second light ray and the third light ray generated by the inner light-emitting element 168 and the outer light-emitting element 170 may be in a wavelength range of near-infrared light. The wavelength of the second light ray is a larger wavelength of a near-infrared light wave band, for example, 940 nanometers. The wavelength of the third light ray is a smaller wavelength in the near-infrared light wave band, for example, 810 nanometers. The second light ray and the third light ray are near-infrared light rays, which causes no discomfort to the user when entering the pupil 41 or can avoid impact on a measurement result when the user uses a mydriatic. The difference in the wavelength of the second light ray and the third light ray enables the physiological information capture device 100 to capture different information of the eye 40 during use.

In some embodiments, when the inner light-emitting element 168 and the outer light-emitting element 170 respectively generate the second light ray and the third light ray to irradiate the eye 40, a plurality of inner light-emitting elements 168 included in a group and a plurality of outer light-emitting elements 170 included in a group are sequentially turned on. For example, four inner light-emitting elements 168 included in a group of inner light-emitting elements 168 are respectively located in four directions of the second hole 164. The inner light-emitting element 168 located at 0 degrees may be first turned on, then the inner light-emitting element 168 located at 90 degrees is turned on, and then the inner light-emitting element 168 located at 180 degrees is turned on, and finally the inner light-emitting element 168 located at 270 degrees is turned on.

When one of the inner light-emitting elements 168 or one of the outer light-emitting elements 170 is turned on, the other inner light-emitting elements 168 or outer light-emitting elements 170 are turned off. If a plurality of groups of outer light-emitting elements 170 are concentrically arranged with respect to the second hole 164, a group of outer light-emitting elements 170 of the plurality of outer light-emitting elements 170 located in an innermost circle are sequentially turned on. After the plurality of outer light-emitting elements 170 included in the group located in the innermost circle each are turned on once, another group of outer light-emitting elements 170 surrounding the innermost circle are sequentially turned on. The operation is repeated until all of the outer light-emitting elements 170 are sequentially turned on.

The second light rays respectively generated after the plurality of inner light-emitting elements 168 are turned on irradiate different positions of the iris 44 and a fundus 46 of the eye 40 of the user, and the third light rays respectively generated after the plurality of outer light-emitting elements 170 are turned on irradiate the pupil 41 of the eye 40 of the user, run through a lens 48 of the eye 40 from the pupil 41, and reach the fundus 46.

Referring to FIG. 7, FIG. 7 is a cross-sectional view of a position A-A marked in FIG. 4. When the second light rays irradiate the iris 44 and the fundus 46 of the eye 40 of the user, the second light rays can run through the first lens 176 of the lens assembly 174. The first lens 176 covers the inner light-emitting elements 168, so that the second light rays can aggregate outside the physiological information capture device 100 through the first lens 176. Specifically, when the user uses the physiological information capture device 100, a part of the second light rays generated by the inner light-emitting element 168 aggregates at a leading edge of the lens 48 of the eye 40 of the user through the first lens 176, and then diverges to the fundus of the eye 46 through the lens 48. Meanwhile, the first lens 176 enables the second light rays to irradiate the iris 44 of the user. If a first mirror 176a of the first lens 176 facing the capturing side 122 is a concave lens, a desirable effect can be achieved, but the present invention is not limited thereto.

In some embodiments, in addition to the first lens 176, the lens assembly 174 may further include a second lens 177 and a third lens 178. A second mirror 177a in the second lens 177 and a third mirror 178a in the third lens 178 are convex lenses, so that the second reflected light of the second light ray reflected from the fundus 46 of the eye 40 can be incident on the second camera module 182.

Moreover, as described above, the included angle θ_{cam} is defined by the COA of the first camera module 180 with respect to the normal line Ln of the upper surface 163a of the second circuit board 162, so that the imaging light ray entering the first camera module 180 can pass through the first hole 144 without being blocked by the first circuit board 142. The “imaging light ray” may be the first reflected light of the first light ray reflected from the sclera 42, the second reflected light of the second light ray reflected from the iris 44, and the third reflected light of the third light ray reflected from the fundus 46.

When the plurality of inner light-emitting elements 168 and the plurality of outer light-emitting elements 170 are sequentially turned on, the first camera module 180 receives the second reflected light of the second light ray or the third reflected light of the third light ray after each turn-on. In this way, the third reflected light generated when the third light rays irradiate different positions of the eye 40 can be obtained.

In some embodiments, the first camera module 180 is located on the upper surface 163a of the second circuit board 162. However, in some other embodiments, the first camera module 180 is located below the second circuit board 162, and a lens thereof is oriented toward the lower surface 163b of the second circuit board 162.

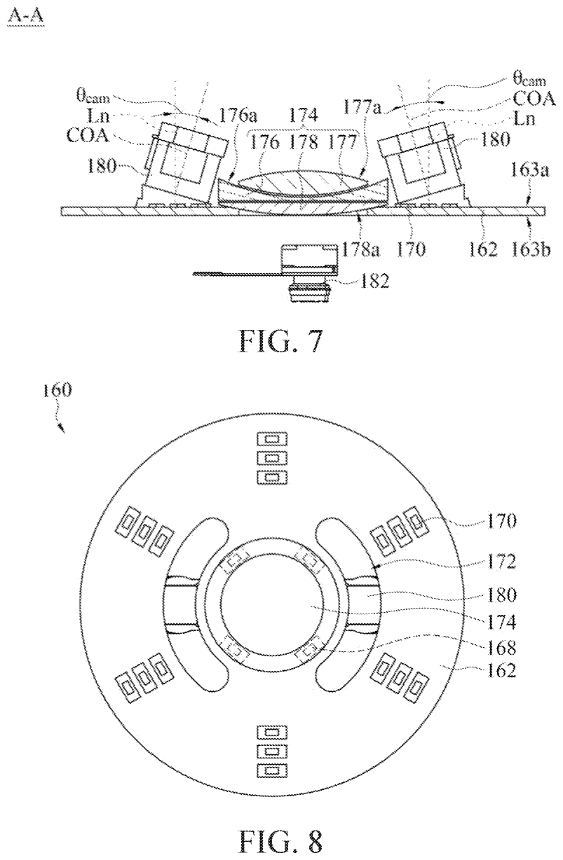

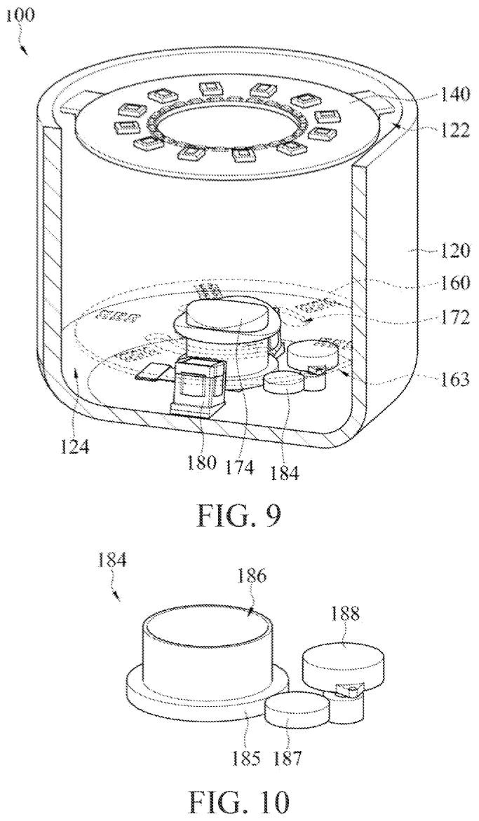

Referring to FIG. 8 to FIG. 10, FIG. 8 is a top view of a second light plate assembly according to an embodiment, FIG. 9 is a three-dimensional schematic diagram of a physiological information capture device according to still another embodiment, showing that the physiological information capture device includes a rotation module, and FIG. 10 is a three-dimensional schematic diagram of an example of the rotation module in FIG. 9.

In some embodiments, to enable the third light rays generated by the second light source module 166 to irradiate more positions of the fundus 46, the physiological information capture device 100 includes a rotation module 184. The rotation module 184 is located in the housing 120 and on the mounting side 124. The second circuit board 162 is provided with openings 172 of a quantity corresponding to a quantity of the first camera modules 180. The openings 172 are located between the inner light-emitting elements 168 and the outer light-emitting elements 170. The openings 172 may be arc-shaped. The rotation module 184 enables the second circuit board 162 to rotate relative to the center of the second hole 164 along a rotation path corresponding to an arc length of the openings 172. The first camera module 180 may receive the first reflected light, the second reflected light, and the third reflected light through the openings 172.

By rotating the second circuit board 162 through the rotation module 184, the third light rays generated by the outer light-emitting elements 170 irradiate varying positions of the fundus 46. Compared with a case in which the second circuit board 162 is relatively fixed, the first camera module 180 can receive third reflected light reflected from more different positions of the fundus 46.

The rotation module 184 may be implemented by a gear shaft 185, a gear 187, and a motor 188. The gear shaft 185 has an accommodating space 186, and the second camera module 182 may be located in the accommodating space 186, so that the second camera module 182 is not affected by the rotation module 184. The lower surface 163b of the second circuit board 162 is fixed to the gear shaft 185, and the second circuit board 162 is rotated by the rotation module 184 in such a way that the motor 188 rotates the gear 187 which, as a result, drives the gear shaft 185 into rotation.

In some embodiments, the first camera module 180 is a multi-spectral camera. Two multi-spectral cameras capture images from two sides of the eye 40. The captured images may be transmitted to the processor 200 for image processing and recovered to a three-dimensional image. The three-dimensional image of the eye 40 is presented by using a second display 350, so that the user can intuitively learn a distribution of physiological substances from the three-dimensional image.

In some embodiments, the second camera module 182 is a liquid lens camera. The liquid lens camera includes a liquid lens and an actuating module. A shape of the liquid lens may be changed by the actuating module to achieve fast focusing. Alternatively, the actuating module may actuate the liquid lens to deform to change a lens optical axis of the liquid lens, so that an image capturing range of the second camera module 182 increases, thereby adapting to photographing of the fundus 46.

In some embodiments, a strap may be connected to an outer side of the housing 120 to form the physiological information capture device 100 as a head-mounted device, which not only facilitates use by a user, but also can achieve smooth data capturing data. In some other embodiments, a desktop fixing device may be connected to the outer side of the housing 120, to form the physiological information capture device 100 as a desktop detection device.

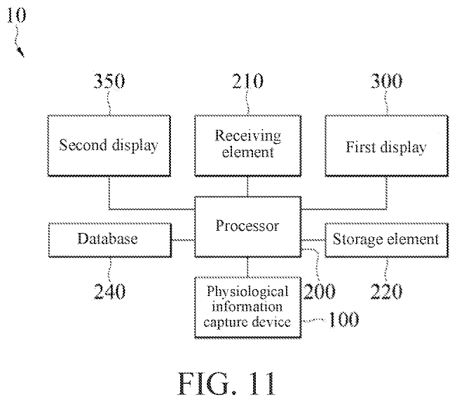

Referring to FIG. 11, FIG. 11 is a block diagram of an ophthalmic examination system according to an embodiment. An ophthalmic examination system 10 includes the foregoing physiological information capture device 100, and further includes a processor 200, a first display 300, and a second display 350. The processor 200 is coupled to the physiological information capture device 100. The first display 300 and the second display 350 are coupled to the processor 200.

The housing 120 of the physiological information capture device 100 may have a connecting line slot. The processor 200 is connected to the first circuit board 142 and the second circuit board 162 of the physiological information capture device 100 through the slot in a form of a connecting line. A format of the connecting line slot is not limited. In addition, a wireless communication module may be arranged in the physiological information capture device 100, and the physiological information capture device 100 may be connected to the processor 200 through wireless communication.

In some embodiments, the ophthalmic examination system 10 includes a receiving element 210. After the user inputs an instruction through the receiving element 210, the receiving element 210 generates a detection instruction. The instruction inputted by the user may be detecting different parts of the foregoing eye 40. After receiving the detection instruction, the processor 200 starts the physiological information capture device 100 based on content of the detection instruction. By using the physiological information capture device 100 and the processor 200, the user may obtain the following information when using the ophthalmic examination system 10: 1) a plurality of physiological values of the physiological substances of the eye 40, 2) a two-dimensional image or a three-dimensional image of a surface of the eye 40, 3) a diopter value of the eye 40, and 4) a fundus image of the fundus 46.

The first display 300 may have a user interface. The user inputs an instruction through the user interface, and the receiving element 210 receives the instruction and then generates a detection instruction. The user interface may have options for selecting a part for detection or options for selecting an item (for example, a physiological substance or a diopter) for detection.

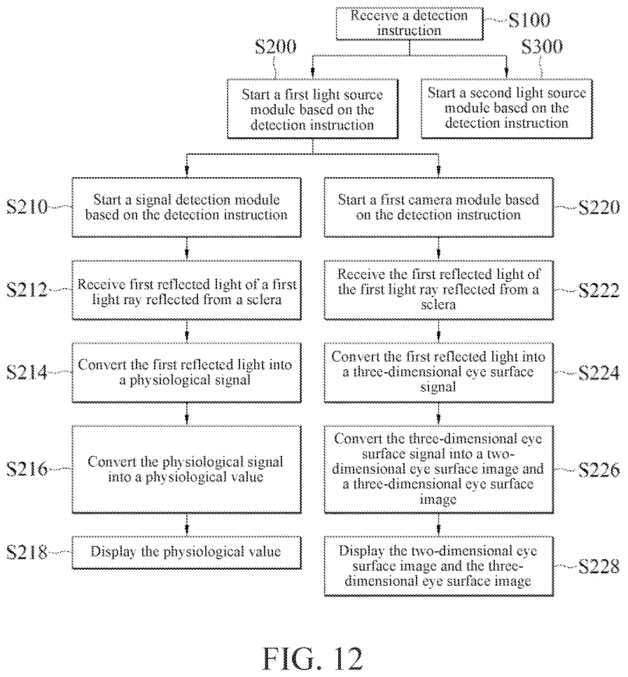

Referring to FIG. 12, FIG. 12 is a flowchart of detecting a physiological value and an eye surface image in an ophthalmic examination method according to an embodiment. When a user desires to obtain a plurality of physiological values of the physiological substances of the eye 40, the processor 200 receives a detection instruction related to detection of the physiological substances (step S100), and starts the first light source module 146 based on the detection instruction (step S200). Each first light-emitting element 150 of the first light source module 146 generates a first light ray to irradiate the sclera 42 of the eye 40. The processor 200 starts the signal detection module 152 based on the detection instruction (step S210). The signal detector 154 of the signal detection module 152 receives first reflected light of the first light ray reflected from the sclera 42 (step S212) and converts the first reflected light into a physiological signal (step S214). The first reflected light received by the signal detector 154 is an optical signal of the first reflected light, and the signal detector 154 may convert the optical signal into an electrical signal (that is, a physiological signal), so that the processor 200 can process the electrical signal.

The first light source module 146 and the signal detection module 152 may irradiate the eye 40 at low power and immediately detect a change in a photoelectric signal of the first light ray within a short period of time through two-second irradiation and two-second receiving.

The processor 200 receives the physiological signal transmitted from the signal detector 154 and then converts the physiological signal into a physiological value (step S216). A type of the physiological value varies with the first light ray generated by the first light-emitting element 150. For example, a wavelength of the first light ray is 460 nanometers. In this case, the physiological value obtained by the processor 200 by converting the physiological signal is a bilirubin value. If the first light rays with different wavelengths, physiological values of different physiological substances may be obtained through conversion. The physiological value is transmitted to the first display 300 and then displayed by the first display 300 (step S218).

Still referring to FIG. 12, when the user desires to obtain a two-dimensional image or a three-dimensional image of a surface of the eye 40, the processor 200 receives a detection instruction related to detection of an eye surface image (step S100), and starts the first light source module 146 based on the detection instruction (step S200). Each first light-emitting element 150 of the first light source module 146 generates a first light ray to irradiate the sclera 42 of the eye 40. Different from the detection of the physiological value, the processor 200 starts the first camera module 180 based on the detection instruction (step S220). The first camera module 180 receives first reflected light of the first light ray reflected from the sclera 42 (step S222), and converts the first reflected light into a three-dimensional eye surface signal (step S224).

The processor 200 receives the three-dimensional eye surface signal transmitted from the first camera module 180 and then converts the three-dimensional eye surface signal into a two-dimensional eye surface image and a three-dimensional eye surface image (step S226). The processor 200 transmits the two-dimensional eye surface image to the first display 300 for display by the first display 300, and transmits the three-dimensional eye surface image to the second display 350 for display by the second display 350 (step S228). In some embodiments, the second display 350 is a 3D naked eye display. Therefore, the two-dimensional eye surface image and the three-dimensional eye surface image can be simultaneously viewed. In some embodiments, the first display 300 displays only the two-dimensional eye surface image, or the second display 350 displays only the three-dimensional eye surface image.

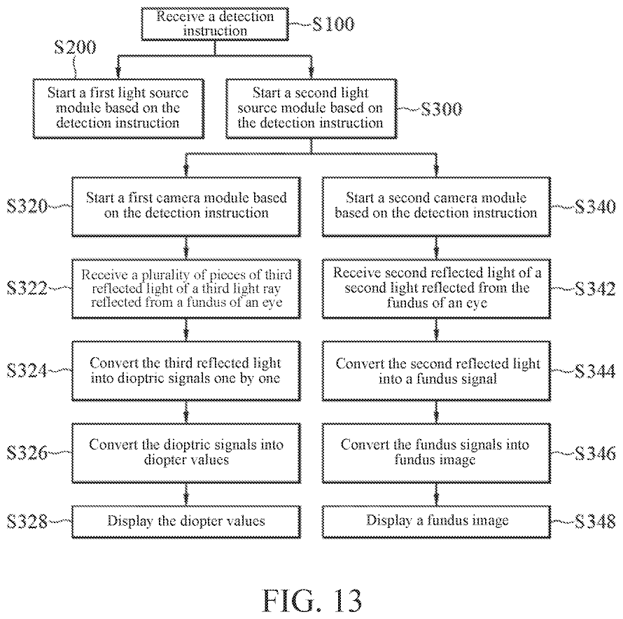

Referring to FIG. 13, FIG. 13 is a flowchart of detecting a diopter value and a fundus image in an ophthalmic examination method according to an embodiment. When a user desires to obtain a diopter value of the eye 40, the processor 200 receives a detection instruction related to detection of a diopter of an eye ball (step S100), and starts the second light source module 166 based on the detection instruction (step S300). The outer light-emitting elements 170 in different directions of the second hole 164 are sequentially turned on one by one to generate third light rays to irradiate the pupil 41 of the eye 40.

To obtain a value of the diopter, the processor 200 starts the first camera module 180 based on the detection instruction (step S320). The first camera module 180 receives, after each of the outer light-emitting elements 170 generates a third light ray, third reflected light of the third light ray reflected from the fundus 46 (step S322). In addition, the plurality of pieces of third reflected light is converted into dioptric signals one by one (step S324). Therefore, a plurality of dioptric signals can be finally obtained.

After receiving the plurality of dioptric signals transmitted from the first camera module 180, the processor 200 converts the dioptric signals into a diopter value through an image operation (step S326). The processor 200 transmits the diopter value to the first display 300 for display by the first display 300 (step S328). The diopter value may provide a degree of myopia/hyperopia of the eye 40 of the user.

Still referring to FIG. 13, when the user desires to obtain a fundus image of the fundus 46, the processor 200 receives a detection instruction related to detection of a fundus image (step S100), and starts the second light source module 166 based on the detection instruction (step S300). Same as the case of the detection of the diopter value, when the second light source module 166 is started, the inner light-emitting elements 168 are also started, to generate second light rays. The second light rays can irradiate the fundus 46 through the lens assembly 174.

Different from the case of the detection of the diopter value, the processor 200 starts the second camera module 182 based on the detection instruction in this case (step S340). The second camera module 182 receives second reflected light of the second light rays reflected from the fundus 46 (step S342), and converts the second reflected light into a fundus signal (step S344). Since the optical axis angle of the second camera module may be adjusted by the actuating module, a plurality of fundus signals before and after the optical axis angle is adjusted can be obtained.

The processor 200 receives the fundus signal transmitted from the second camera module 182. The fundus signal may be composed of a single fundus signal or a plurality of fundus signals. The processor 200 converts the fundus signal into a fundus image through image processing (step S346), and transmits the fundus image to the first display 300 for display by the first display 300 (step S348).

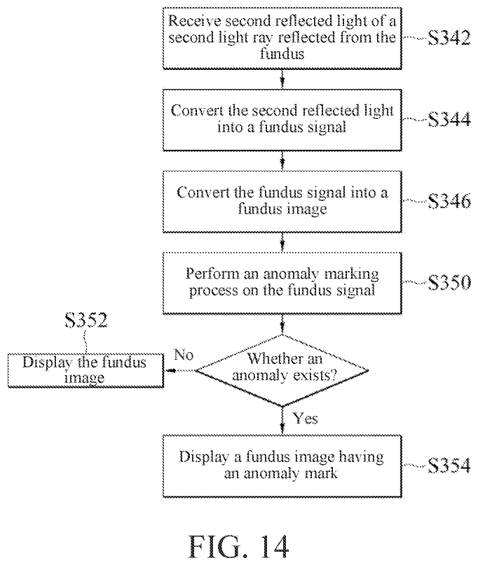

Referring to FIG. 14, FIG. 14 is a flowchart of performing an anomaly marking process in an ophthalmic examination method according to an embodiment. In some embodiments, the processor 200 performs an anomaly marking process on a fundus signal (step S350), so that the first display 300 selectively displays an anomaly mark on the fundus image. The expression “selectively” means that when the processor 200 determines that fundus signals include a signal that conforms to an abnormal condition (for example, a signal with a pathological characteristic), the first display 300 displays a region on the fundus image with an anomaly mark (step S354). If the processor 200 does not recognize the abnormal condition, the processor displays only the fundus image on the first display 300 without marking any anomaly on the visual image (step S352). One or more anomaly marks may be displayed on the fundus image. The anomaly marking process may be performed through an artificial intelligence (AI) neural network operation. The AI neural network operation is established based data in a database 240 and deep learning.

In some embodiments, the ophthalmic examination system 10 includes a storage element 220. The storage element 220 is coupled to the processor 200. The storage element 220 stores the physiological signal, the three-dimensional eye surface signal, the dioptric signal, or the fundus signal received by the processor 200. The storage element 220 may be coupled to the processor 200 wiredly or wirelessly.

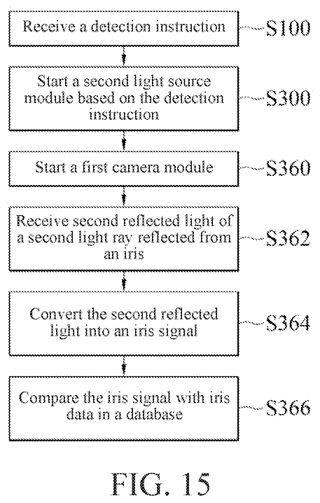

Still referring to FIG. 11 and FIG. 15, FIG. 15 is a flowchart of iris recognition in an ophthalmic examination method according to an embodiment. In some embodiments, the ophthalmic examination system 10 includes a database 240. The database 240 stores a plurality of pieces of iris data. After the processor 200 receives a detection instruction (step S100), the processor may perform iris recognition on a user, and display information corresponding to the user, for example, identity data or a detection record of the user after the recognition. Specifically, during step S100 to step S354, the processor 200 may start the second light source module 166 during any of the steps (step S300).

As described above, when the second light source module 166 is started, the inner light-emitting elements 168 and the outer light-emitting elements 170 are also started to generate second light rays and third light rays. The second light rays can irradiate the iris 44 of the eye 40. The first camera module 180 is started (step S360) to receive second reflected light reflected from the iris 44 (step S362), and then the second reflected light may be converted into an iris signal (step S364).

The iris signal is compared with the plurality of pieces of iris data in the database 240 (step S366), to determine one of the plurality of pieces of iris data corresponding to the iris signal, and the first display 300 outputs information carried in one of the plurality of pieces of iris data corresponding to the iris signal. The information is the identity data or the detection record of the user described above.

In some embodiments, the database 240 is stored in the storage element 220 or in an external device. If the database is stored in the external device, the database 240 may be connected to the processor 200 and the storage element 220 wiredly or wirelessly. When the user uses the ophthalmic examination system 10, a folder dedicated for the user may be opened before the storage element 220 is opened, and the information carried in the iris data corresponding to the user is imported from the database 240 after the iris recognition. Subsequently, the physiological substance, the eye surface image, the diopter, or the fundus image of the user is detected, and the detected physiological signal, three-dimensional eye surface signal, dioptric signal, or fundus signal is stored in the folder of the user.

In some embodiments, the first display 300 may be a common untouchable screen or a touchable screen. The user may select a detection item on a user interface by touching the screen or by using a mouse, a keyboard, or the like.

In some embodiments, the first display 300, the processor 200, the receiving element 210, the storage element 220, and the database 240 may be integrated as an electronic device. The processor 200 is a built-in processor of the electronic device. The storage element 220 is a built-in memory or an external memory of the electronic device. The first display 300 is a screen of the electronic device or an external screen. The electronic device may be a desktop computer, a notebook computer, a laptop computer, a tablet computer, or the like.

In some embodiments, the processor 200 may be, but is not limited to, a central processing unit (CPU), a system on chip (SOC), a general-purpose or special-purpose microprocessor, a digital signal processor (DSP), a programmable controller, an application specific integrated circuit (ASIC), a programmable logic device (PLD), another similar processing device, or a combination of the devices.

In some embodiments, the physiological information capture device 100 may be powered by an adapter, a battery, or a transmission line that obtains power from the outside world. If the battery that supplies power to the device is a rechargeable battery, the rechargeable battery may be charged through the adapter or the transmission line.

Based on the above, after the physiological information capture device 100 is integrated into the ophthalmic examination system 10, the eye 40 is irradiated with light rays of various wave bands (the first light ray, the second light ray, and the third light ray), the reflected light of the light rays of the various wave bands is captured through the signal detection module 152, the first camera module 180, or the second camera module 182, and various physiological or biochemical detection data of the eye 40 may be obtained through the data operation of the processor 20.

Certainly, the present invention may have various other embodiments. Without departing from the spirit of the present invention and its essence, a person skilled in the art may make various corresponding changes and modifications according to the present invention, but these corresponding changes and modifications shall fall within the protection scope of the claims of the present invention.

What is claimed is:

1. A physiological information capture device, comprising:
a housing, comprising a capturing side and a mounting side, wherein the capturing side and the mounting side are opposite to each other;
a first light plate assembly, arranged in the housing and located close to the capturing side, wherein the first light plate assembly comprises:
a first circuit board, provided with a first hole;
a first light source module, located on the first circuit board and surrounding the first hole, wherein the first light source module comprises a plurality of first light-emitting elements; and
a signal detection module, located on the first circuit board, wherein the signal detection module is provided with a plurality of signal detectors corresponding to the plurality of first light-emitting elements; and
a second light plate assembly, arranged in the housing and located close to the mounting side, wherein the second light plate assembly comprises:
a second circuit board, provided with a second hole, wherein the second hole is aligned with the first hole, and a diameter of the first hole is greater than a diameter of the second hole;
a second light source module, located on the second circuit board, wherein the second light source module comprises a group of inner light-emitting elements and at least one group of outer light-emitting elements, the group of inner light-emitting elements surround the second hole, and the at least one group of outer light-emitting elements are concentrically arranged with respect to a center of the second hole and surround the group of inner light-emitting elements and the second hole;
a lens assembly, arranged in the housing, wherein a lens optical axis of the lens assembly is aligned with the center of the second hole, the lens assembly comprises a first lens, and the first lens covers the group of inner light-emitting elements and the second hole;
a plurality of first camera modules, located on a periphery of the second hole, wherein a camera optical axis (COA) of each of the first camera modules defines an included angle relative to a normal line of an upper surface of the second circuit board; and
a second camera module, wherein a COA of the second camera module is aligned with the center of the second hole.

2. The physiological information capture device according to claim 1, wherein the first light source module comprises a plurality of first light source groups with different wavelengths, and each of the first light source groups comprises the plurality of first light-emitting elements with a same wavelength.

3. The physiological information capture device according to claim 1, wherein each of the first light-emitting elements generates a first light ray to irradiate a sclera of an eye, and the plurality of signal detectors receive first reflected light of the first light ray reflected from the sclera of the eye.

4. The physiological information capture device according to claim 3, wherein the group of inner light-emitting elements generate a second light ray to irradiate an iris and a fundus of an eye, the plurality of groups of outer light-emitting elements generate a third light ray to irradiate an pupil of the eye, and the first camera module receives the first reflected light of the first light ray reflected from the sclera of the eye, second reflected light of the second light ray reflected from the iris of the eye, and third reflected light of the third light ray reflected from the fundus of the eye.

5. The physiological information capture device according to claim 4, wherein the second camera module receives the second reflected light of the second light ray reflected from the fundus of the eye.

6. The physiological information capture device according to claim 1, wherein wavelengths of the group of inner light-emitting elements are different from wavelengths of the at least one group of outer light-emitting elements.

7. The physiological information capture device according to claim 1, comprising a rotation module, wherein the second circuit board is provided with a plurality of openings, the plurality of openings are located between the group of inner light-emitting elements and the at least one group of outer light-emitting elements, and the rotation module is enable the second circuit board to rotate relative to the center of the second hole.

8. The physiological information capture device according to claim 1, wherein the first camera module is a multi-spectral camera.

9. The physiological information capture device according to claim 1, wherein the second camera module comprises a liquid lens and an actuating module, and the actuating module actuates the liquid lens to deform to change a focal length of the liquid lens.

10. The physiological information capture device according to claim 9, wherein the actuating module actuates the liquid lens to deform to change a lens optical axis of the liquid lens.

11. An ophthalmic examination system, comprising:
a physiological information capture device, comprising:
a housing, comprising a capturing side and a mounting side, wherein the capturing side and the mounting side are opposite to each other;
a first light plate assembly, arranged in the housing and located close to the capturing side, wherein the first light plate assembly comprises:
a first circuit board, provided with a first hole;
a first light source module, located on the first circuit board and surrounding the first hole, wherein the first light source module comprises a plurality of first light-emitting elements; and
a signal detection module, located on the first circuit board, wherein the signal detection module is provided with a plurality of signal detectors corresponding to the plurality of first light-emitting elements; and
a second light plate assembly, arranged in the housing and located close to the mounting side, wherein the second light plate assembly comprises:
a second circuit board, provided with a second hole, wherein the second hole is aligned with the first hole, and a diameter of the first hole is greater than a diameter of the second hole;
a second light source module, located on the second circuit board, wherein the second light source module comprises a group of inner light-emitting elements and at least one group of outer light-emitting elements, the group of inner light-emitting elements surround the second hole, and the at least one group of outer light-emitting elements are concentrically arranged with respect to a center of the second hole and surround the group of inner light-emitting elements and the second hole;
a lens assembly, arranged in the housing, wherein a lens optical axis thereof is aligned with the center of the second hole, the lens assembly comprises a first lens, and the first lens covers the group of inner light-emitting elements and the second hole;
a plurality of first camera modules, located on a periphery of the second hole, wherein a camera optical axis (COA) of each of the first camera modules defines an included angle relative to a normal line of an upper surface of the second circuit board; and
a second camera module, wherein a COA thereof is aligned with the center of the second hole; wherein
each of the first light-emitting elements generate a first light ray to irradiate a sclera of an eye;
the group of inner light-emitting elements generate a second light ray to irradiate an iris and a fundus of the eye;
the at least one group of outer light-emitting elements generate a third light ray to irradiate a pupil of the eye;
the plurality of signal detectors receive first reflected light of the first light ray reflected from the sclera and convert the first reflected light into a physiological signal;
the first camera modules receive the first reflected light reflected from the sclera, second reflected light of the second light ray reflected from the iris, and third reflected light of the third light ray reflected from the fundus of the eye, and respectively convert the first reflected light, the second reflected light, and the third reflected light into a three-dimensional eye surface signal, an iris signal, and a dioptric signal; and
the second camera module receives the second reflected light of the second light ray reflected from the fundus and convert the second reflected light into a fundus signal;
a processor, coupled to the physiological information capture device and the processor: process the physiological signal and convert the physiological signal into a physiological value, process the three-dimensional eye surface signal and convert the three-dimensional eye surface signal into a three-dimensional eye surface image and a two-dimensional eye surface image, process the dioptric signal and convert the dioptric signal into a diopter value, process the fundus signal and convert the fundus signal into a fundus image, and receive the iris signal;
a first display, coupled to the processor and displays a group composed of the physiological value, the two-dimensional eye surface image, the diopter value, the fundus image, and a combination thereof, and
a second display, coupled to the processor and displays the three-dimensional eye surface image.

12. The ophthalmic examination system according to claim 11, wherein the first light source module comprises a plurality of first light source groups with different wavelengths, and each of the first light source groups comprises the plurality of first light-emitting elements with a same wavelength.

13. The ophthalmic examination system according to claim 11, wherein wavelengths of the group of inner light-emitting elements are different from wavelengths of the at least one group of outer light-emitting elements.

14. The ophthalmic examination system according to claim 11, comprising a rotation module, wherein the second circuit board is provided with a plurality of openings, the plurality of openings are located between the group of inner light-emitting elements and the at least one group of outer light-emitting elements, and the rotation module is enable the second circuit board to rotate relative to the center of the second hole.

15. The ophthalmic examination system according to claim 11, wherein the second camera module comprises a liquid lens and an actuating module, and the actuating module actuates the liquid lens to deform to change a focal length of the liquid lens.

16. The ophthalmic examination system according to claim 11, comprising a storage element, coupled to the processor and stores the physiological signal, the three-dimensional eye surface signal, the iris signal, the dioptric signal, and the fundus signal.

17. The ophthalmic examination system according to claim 11, comprising a database, wherein the processor compares the iris signal with iris data in the database.

18. The ophthalmic examination system according to claim 11, wherein the processor activates the first light source module or the second light source module based on a detection instruction.

19. An ophthalmic examination method, comprising:
receiving a detection instruction;
starting a first light source module or a second light source module based on the detection instruction, wherein the first light source module generates a first light ray, and the second light source module generates a second light ray and a third light ray;
performing when the first light source module is started:
starting a signal detection module or a first camera module based on the detection instruction;
receiving first reflected light of the first light ray reflected from a sclera of an eye;
converting the first reflected light into a physiological signal and converting the physiological signal into a physiological value when the signal detection module is started; and converting the first reflected light into a three-dimensional eye surface signal and converting the three-dimensional eye surface signal into a two-dimensional eye surface image and a three-dimensional eye surface image when the first camera module is started; and
displaying the physiological value or the two-dimensional eye surface image and the three-dimensional eye surface image; and
performing when the second light source module is started:
starting the first camera module or a second camera module based on the detection instruction;
receiving third reflected light of the third light ray reflected from a fundus of the eye, converting the third reflected light into a dioptric signal, converting the dioptric signal into a diopter value, and displaying the diopter value, when the first camera module is started; and
receiving second reflected light of the second light ray reflected from the fundus of the eye, converting the second reflected light into a fundus signal, converting the fundus signal into a fundus image, and displaying the fundus image, when the second camera module is started.

20. The ophthalmic examination method according to claim 19, comprising:
starting the second light source module based on the detection instruction;
starting the first camera module;
receiving the second reflected light of the second light ray reflected from the eye; and
converting the second reflected light into an iris signal, and comparing the iris signal with iris data in a database.
